(19) United States
(12) Patent Application Publication
(10) Pub. No.: US 2020/0005949 A1
(43) Pub. Date: Jan. 2, 2020

(54) Engagement and Education of Patients for Endoscopic Surgery

(71) Applicant: Pristine Surgical, LLC, Manchester, NH (US)

(72) Inventor: Blaine Warkentine, Boulder, CO (US)

(73) Assignee: Pristine Surgical, LLC, Manchester, NH (US)

(21) Appl. No.: 16/278,112

(22) Filed: Feb. 17, 2019

Related U.S. Application Data
(60) Provisional application No. 62/632,829, filed on Feb. 20, 2018.

(51) Int. Cl. G16H 80/00; A61B 1/045; G16H 10/20; G16H 10/60

(52) U.S. Cl. CPC G16H 80/00; A61B 1/045; A61B 2034/107; G16H 10/60; G16H 10/20

(57) ABSTRACT

One or more computers provide an interface that permits a patient to request information about a disease or injury treatable by surgery. The computers host a dialog between the patient and a human expert in treatment and the patient, including taking a patient history for storage into the computer memory. In the event that the patient undergoes surgery, the computers receive a video feed from an endoscope being used in surgery of a patient, and store at least excerpts from the video under control of the surgeon. The computers receive instructions from a member of the surgical team to edit the stored video into an educational video designed to educate the patient in post-surgical care of the surgery site. Via an interface compliant with patient confidentiality laws, the computers provide the educational video to the patient.

### BACKGROUND

This application is a nonprovisional claiming benefit from U.S. Provisional App. Ser. No. 62/632,829, “Engagement and Education of Surgical Patients,” filed Feb. 20, 2018, which is incorporated herein by reference.

This application relates to educating surgery patients in actions that the patient can take to improve outcome and hasten recovery.

### SUMMARY

In general, in a first aspect, the invention features a method, and apparatus designed for performance of the method. During surgery of a patient, one or more computers receive a video feed from an endoscope being used in the surgery. A member of the surgical team that performed the surgery instructs the computers to edit the video into an educational video designed to educate the patient in post-surgical care of the surgery site. The computers present information to the patient via an interface compliant with patient confidentiality laws. The educational video is provided to the patient via this interface.

In general, in a second aspect, the invention features a method, and apparatus designed for performance of the method. One or more computers provide an interface that permits a patient to request information about a disease or injury treatable by surgery. The computers host a dialog between the patient and a human expert in treatment and the patient, including taking a patient history for storage into the computer memory. In the event that the patient undergoes surgery, the computers receive a video feed from an endoscope being used in surgery of a patient, and store at least excerpts from the video under control of the surgeon. The computers receive instructions from a member of the surgical team to edit the stored video into an educational video designed to educate the patient in post-surgical care of the surgery site. Via an interface compliant with patient confidentiality laws, the computers provide the educational video to the patient.

Embodiments of the invention may include one or more of the following features. One or more computers may provide to the patient, access to the edited video, via an interface compliant with HIPAA (Health Insurance Portability and Accountability Act of 1996) patient confidentiality. An endoscope (which may be a laparoscope or arthroscope) for the procedure may include a unique identifier on an object that can be given to the patient, the object including a barcode, QR code, or UDI number assigned by the FDA. Before surgery, one or more of the computers may provide information to the patient to improve the patient's compliance in preparation in surgery. The video feed may originate from a camera at the tip of an endoscope, laparoscope, or arthroscope, as the procedure in progress. The endoscope may have a button or control operable by the surgeon to designate video for inclusion into the edited video. The button or control may be programmable to provide two or more modes of recording, for example, (a) capture an interval of time following a control actuation, (b) capture an interval of time immediately preceding a control actuation, (c) capture an interval of time extending both before and after the control actuation, the next five seconds, (d) capture video during the time when the control is actuated, (e) toggle back and forth between recording and not recording the video, or (f) bookmark a continuously recorded portion of the recorded video. A video editing system of the one or more computers may provide to a person editing the video the ability to provide voice-over annotation of the video. The edited stored video may explain the patient's pre-surgical condition and pathology, what was done during the surgery, the result of the surgery, and any recommendations for post-surgical management, including therapy. The computers may be programmed to provide a video editing capability to place portions of the video, or still frames from the video, side-by-side into the edited video. The editing user interface may provide multiple editing capabilities, including, for example, temporal selection, spatial cropping, incorporation of prerecorded canned clips, juxtaposing two video segments side-by-side, adding voice-over, and adding chalk-drawing markup. The edited video may include educational material helpful to the patient, and/or promotional material from or about the surgeon and/or surgical facility, generic to multiple patients. The educational video may be stored in internet cloud storage, and the viewing interface may provide the educational video to the patient from that cloud storage. The unique identifier may be used as a credential to validate the patient's credentials to view the video. A member of the surgical team that instructs the computer to edit the video may be a person that was not present in the operating room used for the surgery. The computers of the method may include at least one computer in the surgery operating room, and at least one cloud server.

The above advantages and features are of representative embodiments only, and are presented only to assist in understanding the invention. It should be understood that they are not to be considered limitations on the invention as defined by the claims. Additional features and advantages of embodiments of the invention will become apparent in the following description, from the drawings, and from the claims.

### DESCRIPTION OF THE DRAWINGS

FIG. 1a is a block diagram of a computer system.

FIG. 1b is a flowchart.

FIGS. 2a and 2b are screen shots.

FIG. 3 is a schematic view of surgery in progress.

FIGS. 4a, 4b, 5a, 5b, 5c, 6a, and 6b are screen shots.

### DESCRIPTION

The Description is organized as follows.

I. Overview

II. Pre-surgical patient education II.A. Initial patient contact II.B. Pre-surgical counseling and preparation

III. Video segments from endoscopic or arthroscopic surgery

III.A. Capturing video during surgery III.B. Video editing and production III.C. Protection of patient confidential information III.D. Patient viewing of the video, and sharing III.E. Sharing with other physicians III.F. Sharing for perioperative quality control

IV. Educating the patient to prepare for and recover from surgery

V. Alternative embodiments

I. Overview

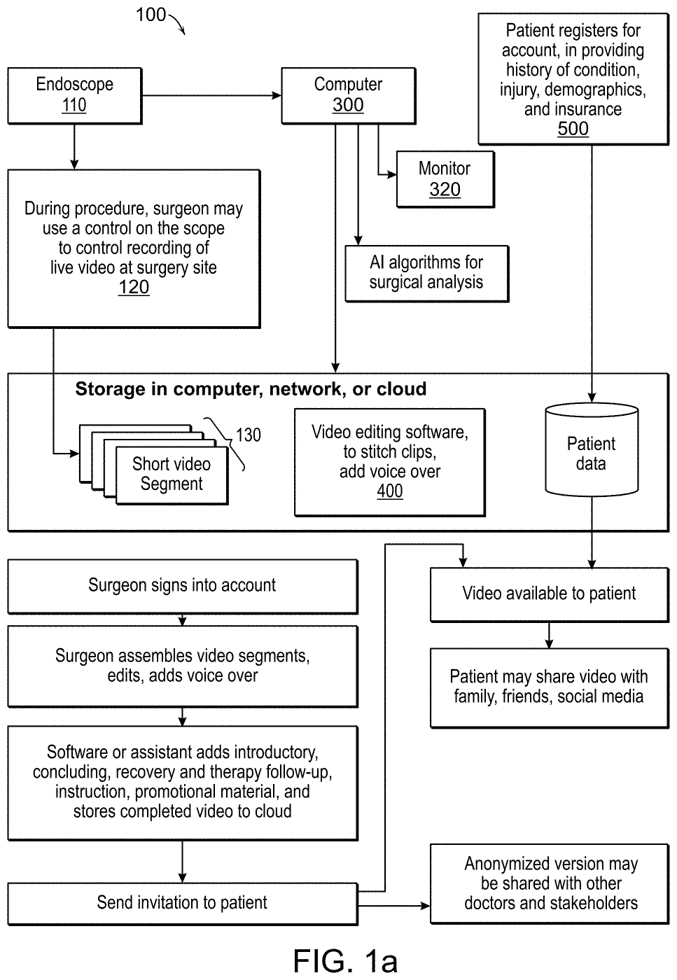

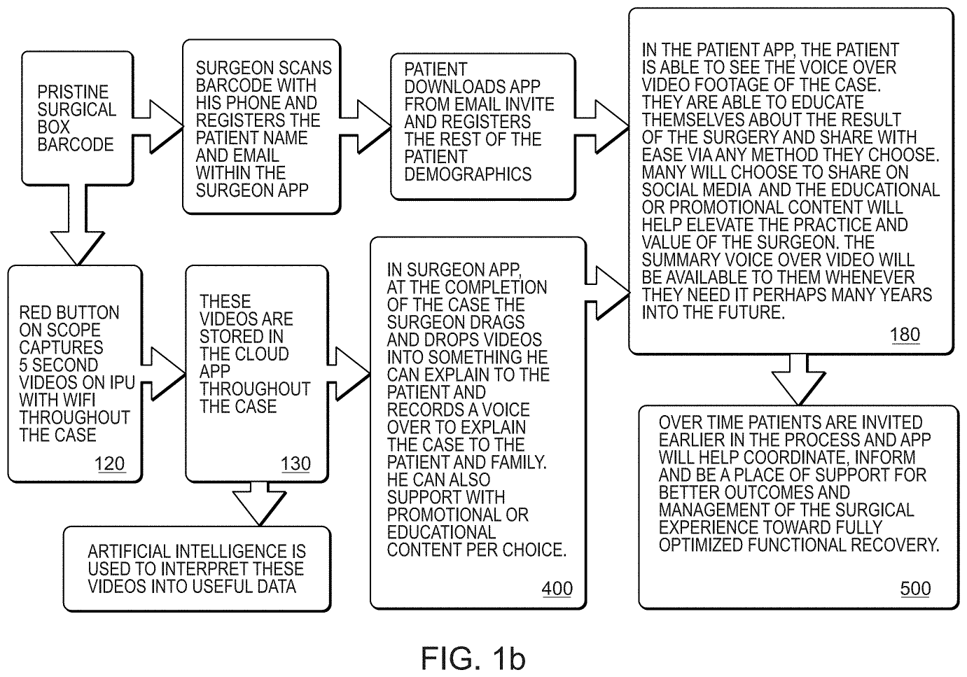

Referring to FIG. 1a, patient outcomes for arthroscopic surgery may be improved when patients and family have properly learned about their condition, treatment, results and recommendations for a successful return to full functional recovery. A cloud-based digital media system 100 may improve communication between a patient and surgeon. Before surgery, surgical education system 100 may gather information 510 about the patient and provide it to the surgeon so that the surgeon can make improved recommendations to the patient. Surgical education system 100 may provide information to the patient to improve the patient's compliance in preparation in surgery (diet, pre-surgical exercise, etc.). During surgery, the surgeon may capture 120 video 130 of portions of the procedure in progress, for example from a camera at the tip of an endoscope 110 (which may be a laparoscope or arthroscope) as the procedure in progress. Post-surgery, the surgeon and medical team may edit 400 video segments 130 into a short film 600 that explains the patient's precise pre-surgical condition and pathology, what was done during the surgery, the result, and any recommendations for post-surgical management, including therapy. Finished video 600 may improve the surgeon's ability to safely and effectively communicate the results of the surgery. Video 600 may have side-by-side before-and-after pictures or video of the tissue or organ operated on, or otherwise communicate a story that communicates the effect of the surgery to the patient, and to family members that assist in post-operative care. Video 600 may include other educational material helpful to the patient, and may include promotional material from or about the surgeon and/or surgical facility. The patient may show and/or share 190 this video or media to family and/or friends. Surgical education system 100 includes protections to ensure the surgeon and facility comply with HIPAA (Health Insurance Portability and Accountability Act of 1996) patient confidentiality, while allowing patients to share their own information as freely as they would like. Patients may be better educated and enthusiastic about what they can do to improve outcomes and speed their own recovery.

About 50% of the overall result of certain classes of surgery can depend on patient diligence and compliance with pre- and post-surgical care. Video may be significantly more effective in communicating patient care information than oral or face-to-face explanations from the surgeon with known poor retention of these conversations of under 10%. Often the most powerful way to motivate the patient is to accentuate the value of actions the patient can take to improve outcome, promptly after surgery. Better educated patients have been shown over and over again to attain better outcomes on average. Patients that understand their disease and treatment are more able to make necessary accommodations and comply with recommendations. Educated patients make better decisions about what kind of activities to engage in following surgery, and when, and how activity choices can improve long-term recovery and bodily function. For example, for patients that have had knee surgery, often one of the most important steps for the patient is to lose weight—patients that understand the need for weight loss are more engaged, and more likely to actually lose the necessary weight. They make better decisions about how much rehab or physical therapy to do. Today, just 17% of American orthopedic surgery patients complete their full prescribed therapy programs, but for patients that are educated about the need for therapy, the rate of completion jumps to about 70%, nearly a five-fold increase. Video of the inside of the patient's own joint may be especially effective in this education. A more-educated, more-engaged patient is more likely to follow through on post-operative therapy, and to be happier with the surgery and surgeon. A video may also be helpful to the patient's family in understanding of what happened during surgery, which may increase family engagement and support.

Referring to FIG. 1b, a surgical instrument as sold or delivered, or a disposable element for the instrument, may be supplied with a card or similar removable element with a unique identifier, such as a barcode, QR code, or UDI number (“unique device identifier” assigned by the FDA). At the beginning of a surgical procedure, the surgeon may scan this bar code, or otherwise associate this unique identifier with the record for the patient and specific procedure. During the procedure, the surgeon may use a button on the scope or a similar trigger to capture video 130 from a camera on the tip of the surgical instrument, to record parts of the surgical procedure. These video segments may be stored either in the computer for the surgical procedure, in the internet cloud, or the like. The surgeon may use a video editing capability 400 to assemble the captured video segments into a presentation for the patient. The surgeon may provide a voice-over annotation of video 600 that explains the procedure and results to the patient and the patient's family. The surgeon may also include educational or promotional content into video 600, and instructions for postoperative therapy. The surgeon may provide the unique identifier to the patient, for example, by including the card with the patient's post-surgical go-home package. The patent, using the barcode or unique identifier, may log in 180 to the system 100 to view video 600. The patient may view video 600, with the surgeon's voice-over.

The patient may educate him/herself about the surgery, the result of the surgery, and postoperative care. The summary voice over video 600 will be available to the patient and family whenever they need it, perhaps many years into the future for future care decisions. The patient may share video 600 with any person he or she chooses. Some patients may choose to share on social media 190. The educational or promotional content may help promote the practice and value of the surgeon. Over time, patients are involved earlier in the process. Video 600 may coordinate care and inform the patient, to support better outcomes and management of the surgical experience toward fully optimized functional recovery.

II. Pre-Surgical Patient Education

II.A. Initial patient contact

A person with a sports injury may have no reliable place to ask questions, and little guidance to select next steps within the health care system. Most patients with sports injuries either ignore the injury and wait for it to heal on its own, or go to an urgent care facility or emergency room. The former can lead to further injury, or delay healing. The latter two (urgent care and emergency rooms) are not well suited to actually treating the patient. Urgent care or an emergency room will typically order a few tests and images, and ask the patient to return—and on return the patient will then typically see a different doctor—so merely diagnosing whether there's a real injury or not can take several weeks, before actual therapeutic treatment begins.

Surgical education system 100 may provide a phone app or an internet web chat service for providing basic medical advice—at least enough to direct a patient to the most appropriate provider. Surgical education system may begin 510 by collecting patient demographic information, and conducting an interview by a chatbot to gather some basic information and route the patient to an appropriate human provider. Surgical education system 100 may then connect the patient via a telemedicine visit with an appropriate physician who can ask further knowledgeable questions, to advise on how to proceed. Because the patient's entire record is stored together, some of the costs of rotating physicians may be reduced.

Surgical education system 100 may be able to advise on steps to take to avoid further injury or surgery. Surgical education system 100 may be able to recommend therapy that may restore the patient without the need for surgery.

If surgical education system 100 (including the human experts) does advise surgery, the referral will reflect more knowledge of the patient and knowledge of a broader spectrum of surgeons and their specialties. Surgeons that have engaged with surgical education system 100 and its patient functions for educating the patient, may be enabled to include the patient in the process, preparing the patient for surgery, and planning recovery after surgery. Surgical education system 100 may be able to be an information focal point for dealing with issues to achieve better medical outcomes, reducing the number of surgeries, in a more comprehensive and effective, and less costly way.

II.B. Pre-surgical counseling and preparation

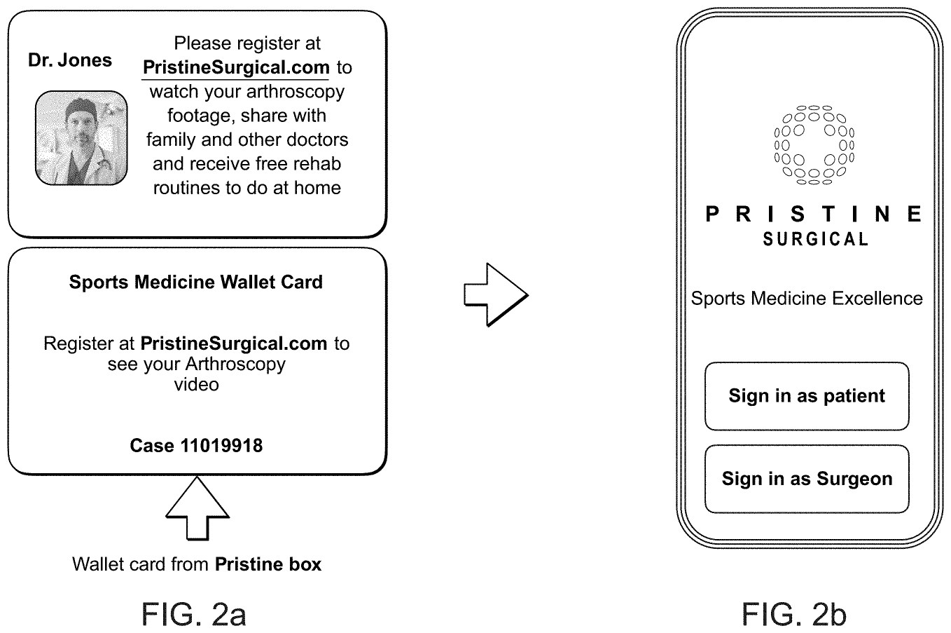

Referring to FIGS. 2a and 2b, surgical education system 100 may engage the patient across an entire episode of care by helping to educate the patient about how to prepare for surgery, and how to recover from surgery. When a patient first approaches a physician, traditionally, the physician creates a record in a conventional electronic medical record system. Additionally, the physician for the initial consultation may create a record for the patient in surgical education system 100, in which the surgeon may provide supplemental annotation, with links back and forth between the conventional electronic medical record system.

Surgical education system 100 may interview 510 the patient, to gather information for the surgeon and surgical team that will be useful in treatment planning, and assist in gathering information for the medical record.

If a surgeon makes a decision to perform surgery on this patient, the surgeon may prepare a pre-surgical educational video for the patient, to educate the patient in pre-surgical preparation. For about two weeks before surgery, surgical education system 100 may advise the patient to adjust diet, exercise regimen, and the like. For the day before surgery, surgical education system 100 may advise the patient to fast and increase liquids, to improve surgical outcome.

III. Video Segments From Endoscopic Or Arthroscopic Surgery

III.A. Capturing video during surgery

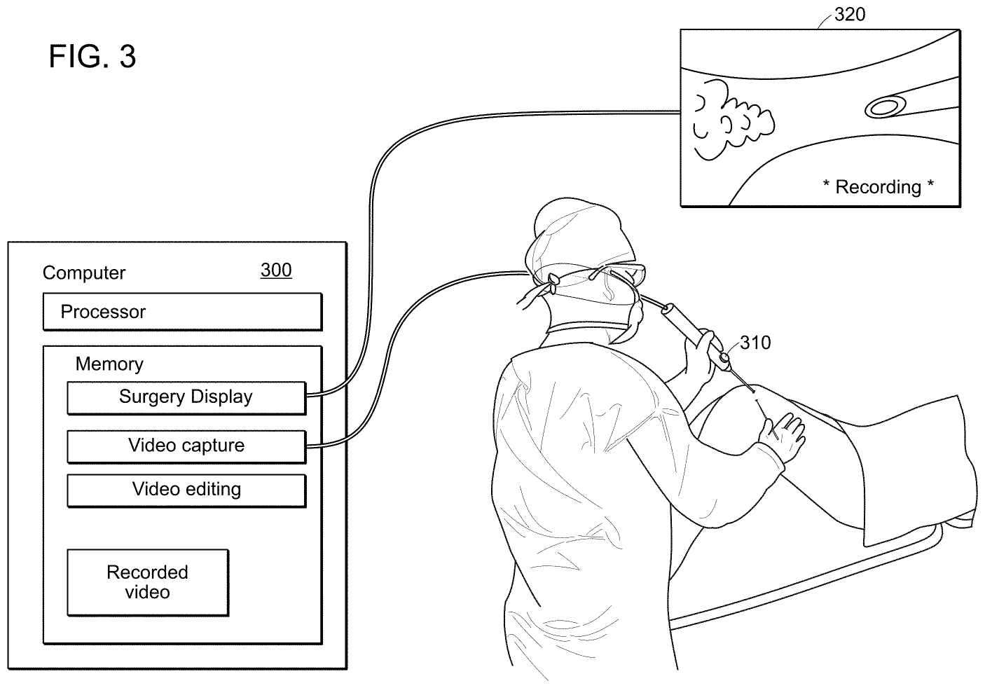

Referring to FIG. 3, endoscopes (including laparoscopes and arthroscopes) often have cameras or fiber optic lenses at or near (1 cm or so) of their tip, to allow a surgeon to see a surgical site within a body. The camera may feed a live video display that is typically displayed on a monitor 320 to guide the surgeon during surgery. The scope or ancillary equipment may be equipped with a button 310, foot pedal, or other actuator to allow the surgeon or clinical staff to control the video, and to effect capture and storage. For example, button 310 may command a computer of surgical education system 100 “capture the next five seconds” or “capture the next ten seconds” or “snapshot the previous ten seconds” or “snapshot ten seconds before and ten seconds after” or “capture video for the period of time the button is depressed” or “toggle back and forth between recording and not recording.”

Alternatively, surgical education system 100 may store the video of the entire procedure, and button 310 may place a “bookmark” that indicates a point of interest, that can be followed up during post-surgical video production. Alternatively, surgical education system 100 may have a touch-sensitive screen with a soft key that can be pressed by one of the staff or assistants when the surgeon gives a voice indication.

The desired operation for the video capture may be programmable by the user. For example, one surgeon may prefer a mode in which a button press captures the next five seconds, while another may program the button to save the previous thirty seconds. A third may prefer to store the entire procedure end-to-end, and use the button to bookmark time points of interest.

If button 310 is programmed to capture a following time window, as surgery proceeds, the surgeon may from time to time pause progress on the procedure itself, and take a moment to use the scope primarily as a camera rather than as an interventional surgical instrument. The surgeon may take a moment to capture some video, and perhaps add a voice-over, to explain the picture—for example, the surgeon may explain video that shows that parts of the organ that are good, video that shows parts that are not and an explanation of the pathology, and then video to show the repair, etc.

III.B. Video editing and production

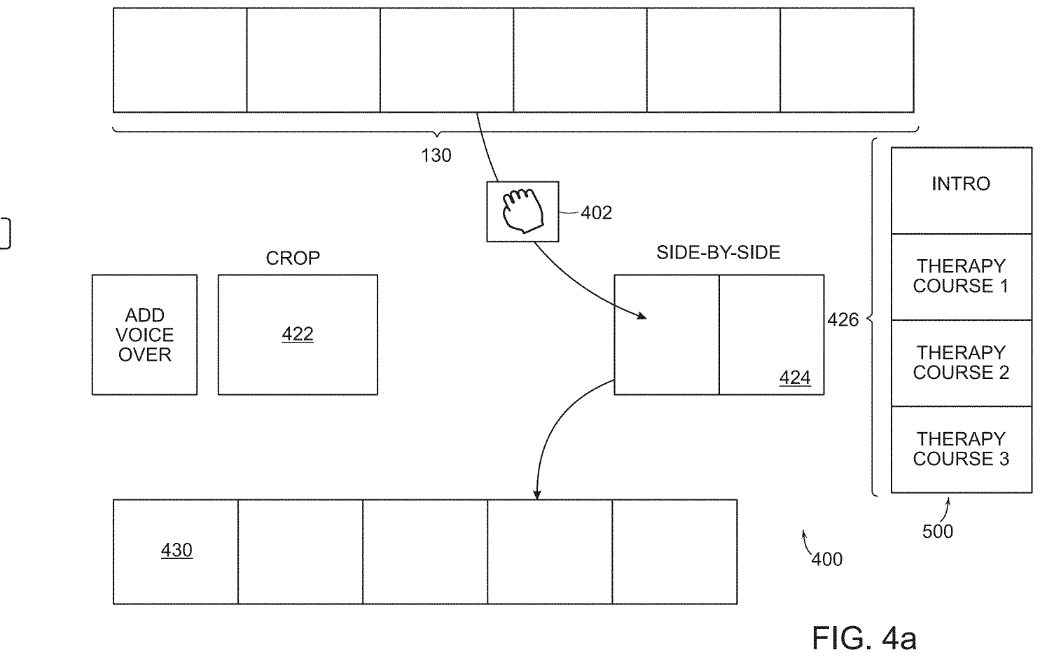

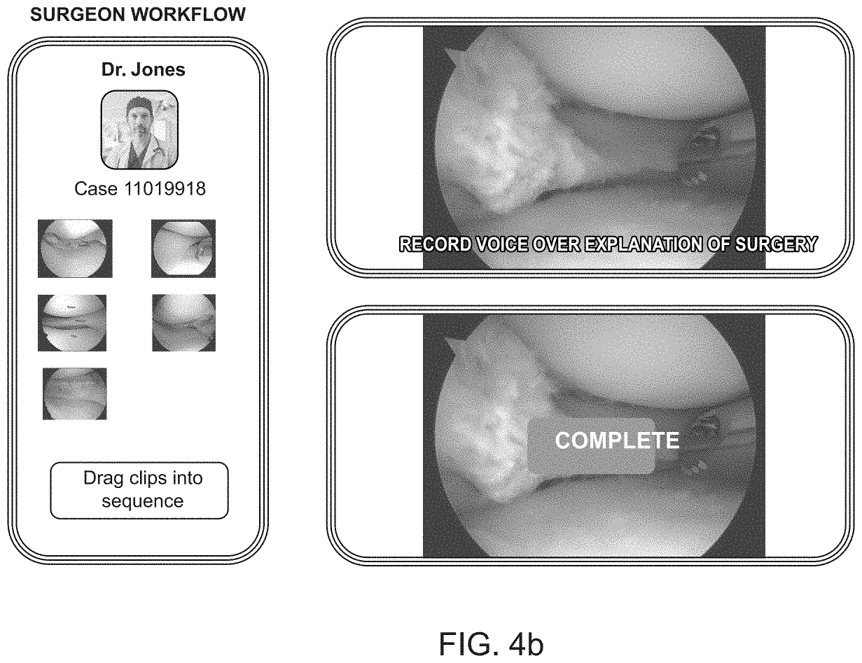

Referring to FIGS. 4a and 4b, after surgery, surgical education system 100 may provide a specialized video editing environment 400. The surgeon may log in to environment 400, and indicate that a specific video is associated with a specific patient.

The surgeon may record the postoperative conference with the family or patient in the waiting room or in a follow-up visit, for use as part of the voice-over in the finished video.

Video editing environment 400 may be tailored around specific kinds of assembly edits that may be most useful for editing raw surgical video segments 130 into finished video 600 for the patient. In one example, the full video of the procedure, or the sequence of five-second raw clips 130 may be arrayed across the top of the screen, to be grabbed by a “hand” to be dragged-and-dropped into stations for editing and assembly.

a “crop” station 422 may take a video clip captured during the procedure, and allow spatial cropping it to fill the frame, or temporal cropping for length

a side-by-side station 424 taking two video clips and/or still frames and juxtaposing them side-by-side to show before and after. When the surgeon wants to juxtapose two scenes, for example, to show a before-and-after contrast, a split screen box in the middle may be used to compose side-by-side clips, which may then, in turn, be dragged to the completed video 430.

a library 426 of “canned” clips for use as an introduction or as a trailer may be available to be edited in

adding voice-over to annotate video 600. Using voice-over, the physician may explain the content of video 600, the condition of the organ before and after surgery, and may explain what features visible in video 600 accounted for pain or other symptoms.

adding chalk-drawing markup (for example, to circle or otherwise highlight specific features) to annotate video 600

a final assembly area 430 may receive each edited segment, and show the assembled video in storyboard form.

The completed video 430 may present selected excerpts in the chronological order from the procedure, or reordered to show certain contrasts. The user interface and available features of video editing environment 400 may be tailored around the kinds of edits that are most likely to help educate the patient, and making those edits easy.

The surgeon may use the voice-over to explain post-surgical care, for example, how much time to take off from work, when and how to resume activities and exercise, and the like.

Editing environment 400 may include “canned” clips 426 to be incorporated into the final video. Examples may include an opening segment, educational material to explain the surgery, explanations and recommendations for post-surgical care and therapy, or promotional material for the surgeon or surgical facility.

Once a finished video 430, 600 is created, it may be stored in a cloud storage location, secured against unauthorized access. The surgeon may provide the patient with access to information and video.

III.C. Protection of patient confidential information

Throughout the process, patient confidential information must be protected to comply with the HIPAA Privacy Rule, under the Health Insurance Portability and Accountability Act of 1996. To comply with those requirements, surgical education system 100 must protect patient confidentiality, and only the patient may authorize disclosure to persons other than the relevant health care professionals. Referring again to FIG. 2a, one way to implement this confidentiality is to include a registration number with any disposable component of the scope. The registration number may be in the form of a numerical or alphanumeric code, bar code, QR code, UDI number, or the like. As surgery begins, the surgeon may associate information (including video clips) with this identification number.

Either before or after surgery, the surgeon may give the patient the identification number, for example, by handing the patient a physical card that was included in the box of disposables for the scope. Providing this information on a single physical object associated with a specific device assures that disclosure will be confined to a specific patient. The identification number may be provided in other channels, as well.

III.D. Patient viewing of the video, and sharing

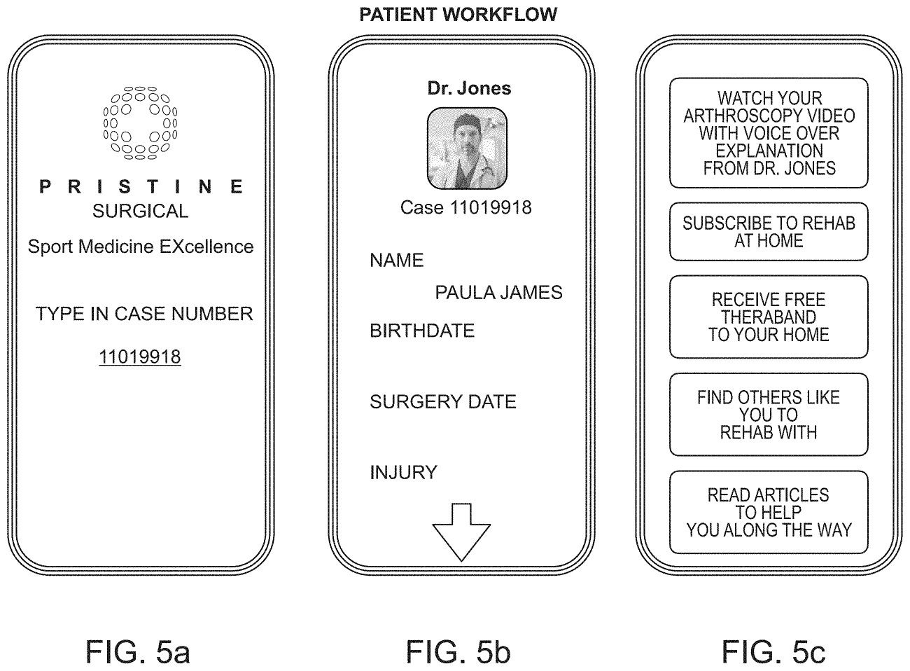

Referring to FIGS. 5a, 5b, and 5c, this identification number may permit the patient to register 510 into surgical education system 100. To complete registration, surgical education system 100 may require the patient to provide additional identification information, to ensure that medical information will only be shared with the patient and those authorized by the patient.

Once logged in, the patient may have access to all information and completed video that the surgeon has uploaded into surgical education system 100 for this particular registration number.

Completed video 600 may provide the patient with a clearer idea of the condition that led to surgery, why the surgery was done, what surgical procedure accomplished. Video 600 may advise on post-surgical care, therapy, and return to normal activity.

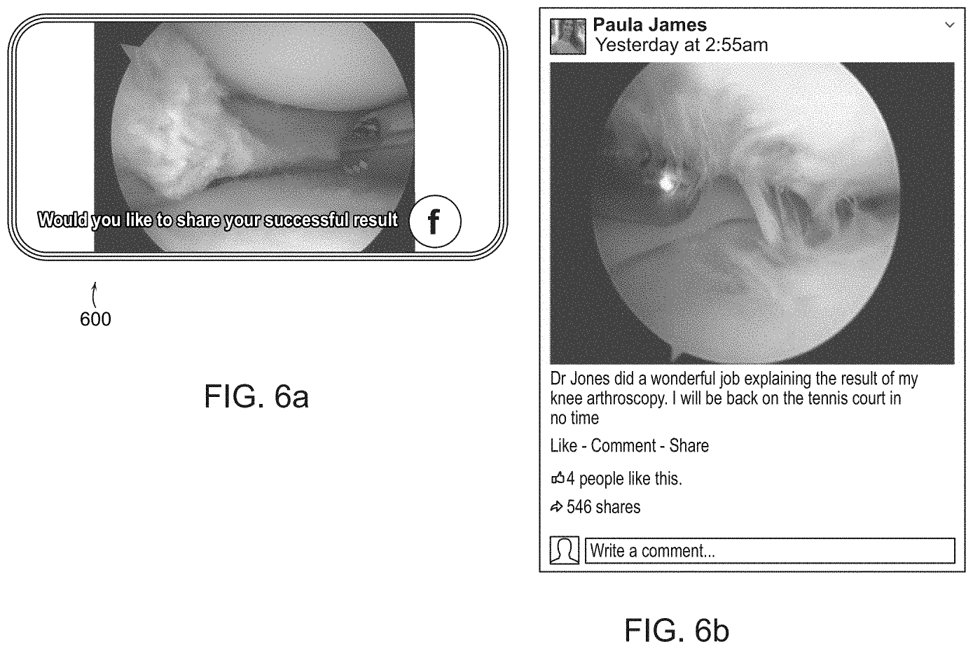

Referring to FIG. 6a, once the patient has registered and logged in and has had identification verified, the patient has discretion to share their video 600 and results as they please. The patient may choose to share their video and other information with a physical therapist or other post-surgical treatment provider.

The patient may choose to share their video and other information with a family member or friend. The patient may choose to share more broadly, for example on Facebook. This may improve connection with friends, to indicate progress to them and estimate return to normal life activities. Friends may respond with sympathy, or offer a game of tennis when recovery is complete.

Video 600 and other information may be stored more or less indefinitely, to be available in case of further surgery.

III.E. Sharing with other physicians

Video 600 may be shared with other physicians and surgeons for educational purposes. For example, longer excerpts from the raw video 130 (up to the entire procedure) may be useful to illustrate technique, intra-procedure adaptation or crisis management, and the like. The entire video 600 may be stored to the cloud, where it may be streamed to other physicians.

To maintain HIPAA compliance, video shared with anyone other than the patient may be anonymized by dissociating any personally-identifiable information such as name or medical record number.

III.F. Sharing for perioperative quality control

Video 600 may be valuable for medical legal reasons, and for perioperative assessment. Even though everyone has good intentions, some orthroscopic surgery achieves little patient benefit, often because the surgeon did not have sufficient information about the patient's morbidity or surrounding life to assess suitability of surgery. The result is that often surgeries are done on people that didn't need them. Several large randomized controlled studies have shown that when patients are randomly assigned to receive either a sham incision versus full surgical treatment, outcomes are the same—patients do just as well with either procedure. E.g., J. B. Moseley et al., A Controlled Trial of Arthroscopic Surgery for Osteoarthritis of the Knee, N Engl J Med 2002; 347:81-88 DOI: 10.1056/NEJMoa013259 (Jul. 11, 2002) (“the outcomes after arthroscopic lavage or arthroscopic débridement were no better than those after a placebo procedure.”); Raine Sihvonen et al. Arthroscopic Partial Meniscectomy versus Sham Surgery for a Degenerative Meniscal Tear, N Engl J Med 2013 369;26 DOI: 10.1056/NEJMoa1305189.

Video of surgical procedures may be used to evaluate procedures. Video 600 may be evaluated via artificial intelligence, an insurer, or some third party. That evaluation of video 600 may be combined with other pre- and post-surgical information and patient assessment (for example, pain, change in activities of daily living, sports, and the like) to evaluate the surgery and the perioperative evaluation that preceded it ex post. That combined analysis and other outcome metrics may be used to develop better ex ante guidelines for appropriateness of care. Various stakeholders such as public health authorities, health insurers, and the like may receive the data to evaluate appropriateness and effectiveness of care. In some cases, insurers may increase reimbursement levels for physicians that provide this information, to compensate for the more extensive pre-surgical work-up and better ex ante perioperative evaluation. Over time, this could change the dynamics on over-utilization of arthroscopy.

IV. Educating the Patient to Prepare For and Recover From Surgery

For days to weeks to months (depending on the nature of the surgery) various post-operative steps may improve outcomes, lead to more complete recovery, and reduce the need for future surgery. Surgical education system 100 may recommend therapy routines day-by-day, and receive reports from the patient in the form of a diary showing what the patient actually did day-by-day, so that surgical education system 100 can monitor compliance with physical therapy, and correlate that to improved function and recovery. As surgical education system 100 learns from multiple patients, machine learning techniques may be used to improve recommendations.

Surgical education system 100 may provide a chat facility. Some questions can be answered by an intelligent digital conversation bot. Other questions may be referred to a human such as a skilled specialist nurse, who can answer questions and offload the surgeon.

Surgical education system 100 may be designed to help design and recommend rehabilitation and physical therapy routines for the pre-surgical preparation, and for post-surgical recovery. These recommendations may speed the patient's return to desired activities, such as participation in sports.

In sports medicine, the goal is to return the patient to activity, maybe even high level activity. For professional athletes, the goal is to return the patient to pitching, throwing, running, or the like. Non-professional athletes wish to return to running, and jogging, skiing, or tennis. Sports medicine surgery seldom involves life-threatening injury; the goal is return to activity. This presents additional opportunities, because activity is relatively easy to measure, and measurement can drive treatment decisions. A patient interacting with surgical education system 100, after reporting an injury, may begin to wear an activity meter, such as a FitBit or similar activity tracker or monitor. Surgical education system 100 may help a physician understand the patient's condition, activity levels, and the like. This knowledge may guide treatment decisions. Also, the monitor may provide real time feedback, allowing treatment to adjust. The monitor may allow before-and-after comparisons to help evaluate the effectiveness of surgery.

V. Alternative Embodiments

Various processes described herein may be implemented by appropriately programmed general purpose computers, special purpose computers, and computing devices. Typically a processor (e.g., one or more microprocessors, one or more microcontrollers, one or more digital signal processors) will receive instructions (e.g., from a memory or like device), and execute those instructions, thereby performing one or more processes defined by those instructions. Instructions may be embodied in one or more computer programs, one or more scripts, or in other forms. The processing may be performed on one or more microprocessors, central processing units (CPUs), computing devices, microcontrollers, digital signal processors, or like devices or any combination thereof. Programs that implement the processing, and the data operated on, may be stored and transmitted using a variety of memory media. In some cases, hard-wired circuitry or custom hardware may be used in place of, or in combination with, some or all of the software instructions that can implement the processes. Algorithms other than those described may be used.

Programs and data may be stored in various media appropriate to the purpose, or a heterogenous combination of media that may be read and/or written by a computer, a processor or a like device. The media may include non-volatile media, volatile media, optical or magnetic media, dynamic random access memory (DRAM), static ram, a floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH-EEPROM, any other memory chip or cartridge or other memory technologies. Transmission media include coaxial cables, copper wire and fiber optics, the wires that comprise a system bus coupled to the processor, and various wireless media.

Databases may be implemented using database management systems or ad hoc memory organization schemes. Alternative database structures to those described may be readily employed. Databases may be stored locally or remotely from a device which accesses data in such a database.

In some cases, the processing may be performed in a network environment including a computer that is in communication (e.g., via a communications network) with one or more devices. The computer may communicate with the devices directly or indirectly, via any wired or wireless medium (e.g., the Internet, LAN, WAN or Ethernet, Token Ring, a telephone line, a cable line, a radio channel, an optical communications line, wifi, commercial on-line service providers, bulletin board systems, a satellite communications link, a combination of any of the above). Each of the devices may themselves comprise computers or other computing devices, such as those based on the Intel® Pentium® or Centrino™ processor, that are adapted to communicate with the computer. Any number and type of devices may be in communication with the computer.

A server computer or centralized authority may or may not be necessary or desirable. In various cases, the network may or may not include a central authority device. Various processing functions may be performed on a central authority server, one of several distributed servers, or other distributed devices

For the convenience of the reader, the above description has focused on a representative sample of all possible embodiments, a sample that teaches the principles of the invention and conveys the best mode contemplated for carrying it out. Throughout this application and its associated file history, when the term “invention” is used, it refers to the entire collection of ideas and principles described; in contrast, the formal definition of the exclusive protected property right is set forth in the claims, which exclusively control. The description has not attempted to exhaustively enumerate all possible variations. Other undescribed variations or modifications may be possible. Where multiple alternative embodiments are described, in many cases it will be possible to combine elements of different embodiments, or to combine elements of the embodiments described here with other modifications or variations that are not expressly described. A list of items does not imply that any or all of the items are mutually exclusive, nor that any or all of the items are comprehensive of any category, unless expressly specified otherwise. In many cases, one feature or group of features may be used separately from the entire apparatus or methods described. Many of those undescribed variations, modifications and variations are within the literal scope of the following claims, and others are equivalent.

The invention claimed is:

1. A method, comprising the steps of:
at a computer, receiving and recording a video feed from a camera at or near a tip of an endoscope being used in surgery of a patient;
receiving instructions at the computer to edit the recorded video into an educational video for the patient, via a user interface designed to ease editing the recorded video into an educational video having form and annotation content designed to educate the patient in post-surgical care, and performing the received instructions to generate and store the educational video into a memory of the one or more computers; and
via a viewing interface compliant with patient confidentiality laws, providing the educational video to the patient.

2. The method of claim 1, further comprising the steps of:
by one or more computers, receiving an initial inquiry from a patient requesting information about a disease or injury treatable by surgery; and
by the one or more computers, moderating a dialog between the patient and a human expert in treatment of the patient's disease or injury, including taking a patient history for storage into the computer memory.

3. The method of either of claim 1, further comprising the step of:
performing the surgery with an endoscope having a button or control operable by the surgeon to designate video for inclusion into the educational video.

4. The method of claim 3, in which:
the endoscope has a control designed to control recording of the received video, the system providing at least three modes of operation of the control, the modes including at least two of: (a) capture an interval of time following a control actuation, (b) capture an interval of time immediately preceding a control actuation, (c) capture an interval of time extending both before and after the control actuation, the next five seconds, (d) capture video during the time when the control is actuated, (e) toggle back and forth between recording and not recording the video, or (f) bookmark a continuously recorded portion of the recorded video.

5. The method of claim 1, in which:
the editing user interface provides three or more capabilities, including at least two of temporal selection, spatial cropping, incorporation of prerecorded canned clips, juxtaposing two video segments and/or still frames side-by-side, adding voice-over, and adding chalk-drawing markup.

6. The method of claim 1, in which:
the educational video includes an explanation of the patient's pre-surgical condition and/or pathology, what was done during the surgery, the result, and recommendations for post-surgical management, in each case, specific to the patient.

7. The method of claim 1, in which:
the educational video includes educational material helpful to the patient, and/or promotional material from or about the surgeon and/or surgical facility, in either case, generic to multiple patients.

8. The method of claim 1, in which:
the educational video is stored in internet cloud storage, and the viewing interface provides the educational video to the patient from that cloud storage.

9. The method of claim 1, further comprising the step of:
providing to the patient an object bearing a unique identifier for the endoscope used in the surgery, and
receiving that unique identifier as part of the credential verification of the patient to establish the patient's right to view the video.

10. The method of either of claim 1, further comprising the step of:
before surgery, by one or more of the computers, providing information to the patient to improve the patient's compliance in preparation in surgery.

11. A system, comprising:
one or more computers, each having a processor and a memory, the one or more memories having stored therein one or more programs designed to cause the computer(s) to:
receive and record a video feed from a camera at or near a tip of an endoscope being used in surgery of a patient;
receive instructions to edit the recorded video into an educational video for the patient, via a user interface designed to ease editing of the recorded video into an educational video having form and annotation content designed to educate the patient in post-surgical care, and to perform the received instructions to generate and store the educational video into a memory of the one or more computers; and
provide a viewing interface compliant with patient confidentiality laws, by which to provide the educational video to the patient.

12. The system of claim 11, the programs being further programmed to cause the computer(s) to:
receive an initial inquiry from a patient requesting information about a disease or injury treatable by surgery; and
moderate a dialog between the patient and a human expert in treatment of the patient's disease or injury, including taking a patient history for storage into the computer memory.

13. The system of either of claim 11, in which:
the endoscope has a button or control operable by the surgeon to designate video for inclusion into the educational video.

14. The system of claim 13, in which:
the endoscope has a control designed to control recording of the received video, the system providing at least three modes of operation of the control, the modes including at least two of: (a) capture an interval of time following a control actuation, (b) capture an interval of time immediately preceding a control actuation, (c) capture an interval of time extending both before and after the control actuation, the next five seconds, (d) capture video during the time when the control is actuated, (e) toggle back and forth between recording and not recording the video, or (f) bookmark a continuously recorded portion of the recorded video.

15. The system of claim 11, in which:
the editing user interface is programmed to provide three or more capabilities, including at least two of temporal selection, spatial cropping, incorporation of prerecorded canned clips, juxtaposing two video segments and/or still frames side-by-side, adding voice-over, and adding chalk-drawing markup.

16. The system of claim 11, in which:
the educational video includes an explanation of the patient's pre-surgical condition and/or pathology, what was done during the surgery, the result, and recommendations for post-surgical management, in each case, specific to the patient.

17. The system of claim 11, in which:
the educational video includes educational material helpful to the patient, and/or promotional material from or about the surgeon and/or surgical facility, in either case, generic to multiple patients.

18. The system of claim 11, in which:
the educational video is stored in internet cloud storage, and the viewing interface provides the educational video to the patient from that cloud storage.

19. The system of claim 11, in which:
the endoscope is supplied including an object bearing a unique identifier for the endoscope used in the surgery, the object being designed to be presented to the patient, and
the programs being further programmed to cause the computer(s) to receive that unique identifier as part of the credential verification of the patient to establish the patient's right to view the video.

20. The system of claim 11, in which the programs are further programmed to:
before surgery, provide information to the patient to improve the patient's compliance in preparation in surgery.
